(19) United States
(12) Patent Application Publication
(10) Pub. No.: US 2014/0221842 A1
(43) Pub. Date: Aug. 7, 2014

(54) System and Method for Frequency Domain Photoacoustic Intravascular Imaging

(71) Applicants: Robin F. Castelino, Toronto (CA); F. Stuart Foster, Toronto (CA)

(72) Inventors: Robin F. Castelino, Toronto (CA); F. Stuart Foster, Toronto (CA)

(21) Appl. No.: 14/169,435

(22) Filed: Jan. 31, 2014

Related U.S. Application Data
(60) Provisional application No. 61/759,835, filed on Feb. 1, 2013.

(51) Int. Cl. A61B 5/00; A61B 8/12; A61B 8/00

(52) U.S. Cl. CPC A61B 5/0095; A61B 8/4416; A61B 8/12
USPC 600/459; 600/407

(57) ABSTRACT

A photoacoustic device capable of performing both intravascular photoacoustic (“IVPA”) imaging and intravascular ultrasound imaging is provided. The device includes one or more optical fibers coupled to a transducer assembly that includes an photoacoustic transducer and optionally an ultrasound transducer. The one or more optical fibers and photoacoustic transducer are arranged such that the illumination field generated by the one or more optical fibers is co-aligned with the sensitive region of the photoacoustic transducer. When both are present, the photoacoustic transducer and ultrasound transducer are arranged such that ultrasound generated by the ultrasound transducer avoids interfering with the sensitive region of the photoacoustic transducer. Frequency domain imaging may be achieved using an intensity-modulated continuous wave (“CW”) laser coupled to the one or more optical fibers.

### CROSS-REFERENCE TO RELATED APPLICATIONS

This application claims the benefit of U.S. Provisional Patent Application Ser. No. 61/759,835, filed on Feb. 1, 2013, and entitled “System and Method for Frequency Domain Photoacoustic Intravascular Imaging.”

### BACKGROUND OF THE INVENTION

The field of the invention is systems and methods for photoacoustic imaging. More particularly, the invention relates to systems and methods for intravascular photoacoustic (“IVPA”) imaging using an intensity modulated continuous wave (“CW”) laser to generate images by a frequency-domain imaging technique.

Photoacoustic (“PA”) imaging is a hybrid imaging technology that combines the strengths of both optical and ultrasound imaging. In PA imaging, nanosecond laser pulses are used to cause local temperature rises in an object. The local temperature rises are accompanied by quick thermal expansion in the object, which is selectively absorbed according to the properties of the object materials. For instance, the object may be tissue and the tissue components such as blood vessels and interstitium can absorb the thermal energy differently. The rapid absorption of the thermal energy causes a thermo-elastic response that generates ultrasound waves that are detected by conventional ultrasound means.

This imaging technique is in contrast to most optical techniques where imaging is effectively limited to reflectivity of ballistic photons and therefore is limited to depths less than two millimeters. Because PA methods are not reliant on ballistic photons, the absorption processes can occur far deeper in tissue and the resultant images can be obtained with spatial resolutions common to ultrasound imaging.

Two principal methods of PA imaging have been developed. The first general class of PA imaging is optical resolution photoacoustic microscopy (“OR-PAM”), in which co-focused optical and ultrasound beams define a system resolution approaching that of optical imaging alone. These systems permit micron level resolution at depths that exceed the limits of competing optical imaging technologies, nonetheless, OR-PAM is still comparatively superficial and not feasible in imaging of deeper tissues.

The second general class of PA imaging is photoacoustic tomography (“PAT”). PAT systems are more suitable for larger tissue volumes and rely on diffuse optical illumination combined with tomographic reconstruction. PAT systems utilize a single ultrasound detector that must be moved to image a volume-of-interest; an array of ultrasound detectors, each with an acceptance angle covering the volume-of-interest; or a combination of the two. Because scanning a single transducer over a volume-of-interest is time intensive, the use of a detector array is highly desirable, especially when combining the optical sources with existing array designs.

Both classes of PA imaging have traditionally been demonstrated using a pulsed optical approach of illumination; that is, using a light source that uses a pulsed laser. Recently, however, a new paradigm for photoacoustic imaging that involves the use of modulated continuous wave (“CW”) lasers and frequency domain processing to create the PA image was described in US Patent Application No. 2005/0234319. The use of frequency domain processing ameliorates many of the issues listed above and may offer the potential to produce a commercial system at reduced cost and with improved performance. The major motivation for this method, referred to as frequency-domain photoacoustics (“FDPA”), is the availability of compact and inexpensive CW laser diodes with a wide wavelength selection in comparison to bulky and expensive Q-switched pulsed lasers, thereby raising the possibility for portable, sensitive PA imagers.

Intravascular ultrasound (“IVUS”) imaging is an established technology and is frequently used for diagnostic imaging and guidance protocols in interventional procedures. In IVUS imaging of coronary arteries, a single element or array-based IVUS catheter is inserted into the lumen of the artery to produce a real-time, high-resolution image of the vessel wall. Although, IVUS imaging can delineate thickness of the vessel and certain structures within the vessel wall, it is generally reported to have low sensitivity in the detection of thrombus and lipid-rich lesions due to the limited acoustic contrast among different types of soft tissues. Due to this limitation of low contrast in soft tissues, other techniques need to be used which do not rely purely on acoustic backscatter of tissue as the contrast mechanism.

By combining photoacoustics with IVUS it is possible to selectively image different soft tissues and lipids by their light absorption characteristics. Using this method, an IVUS transducer can detect PA signals generated by light delivered using one or many optical fibers in a technique known as intravascular photoacoustic (“IVPA”). Current IVPA devices use a pulsed laser system where mostly a nanosecond light pulse is used to illuminate the region of interest. The first bench-top system combining pulsed IVPA/IVUS was demonstrated in US Patent Application No. 2011/0021924, showing the feasibility of this imaging modality on tissue-mimicking phantoms and ex vivo vessel models. Imaging frame-rates, however, were limited to a few Hertz due to the slow repetition rates of pulsed nanosecond lasers. This is a severe limitation for cardiovascular applications, such as preclinical heart imaging or IVUS imaging in humans, where motion of vascular structures can be significantly large. Also, the high cost of pulsed lasers and their inherent instability hinders the acceptance of this technique.

It would therefore be desirable to provide an intravascular photoacoustic device that is capable much higher frame rates than existing devices by using frequency-domain photoacoustic imaging together with intravascular ultrasound imaging.

### SUMMARY OF THE INVENTION

The present invention overcomes the aforementioned drawbacks by providing a photoacoustic device for imaging the lumen of an organ, such as a blood vessel, or other organs, such as the prostate via the urethra or the esophagus via the oral tract. Thus, the photoacoustic device may, in some configurations, be referred to as an intravascular photoacoustic (“IVPA”) imaging device.

It is an aspect of the invention to provide a photoacoustic imaging device that includes a laser configured to generate laser light, at least one optical fiber, and a transducer assembly. The at least one optical fiber extends from a proximal end to a distal end along a longitudinal axis and is optically coupled at its proximal end with the continuous wave laser so as to generate an illumination field at its distal end. The transducer assembly extends from a proximal end to a distal end along a longitudinal axis and is coupled to the at least one optical fiber. The transducer assembly includes a photoacoustic transducer arranged at the distal end of the transducer assembly. The photoacoustic transducer is configured such that a field-of-view of the photoacoustic transducer is co-aligned with the illumination field and to receive photoacoustic emissions generated by the illumination field in the field-of-view.

It is another aspect of the invention to provide a combined photoacoustic and ultrasound imaging device that includes a fiber assembly that is coupled to a transducer assembly. The fiber assembly includes at least one optical fiber that extends from a proximal end to a distal end along a longitudinal axis. The fiber assembly is configured to optically couple the proximal end of the at least one optical fiber to a light source so the at least one optical fiber receives light and generates an illumination field at the distal end. The transducer assembly includes an photoacoustic transducer and an ultrasound transducer. The photoacoustic transducer is configured to receive photoacoustic emissions generated in a field-of-view that is co-aligned with the illumination field. The ultrasound transducer is configured to generate an ultrasound emission field that extends along a direction from the ultrasound transducer such that the ultrasound emission field avoids interfering with the field-of-view.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an example of a photoacoustic imaging device configured for photoacoustic imaging of the lumen of an organ, such as a blood vessel;

FIG. 2 is an example of a photoacoustic imaging device configured for photoacoustic and ultrasound imaging of the lumen of an organ, such as a blood vessel; and

FIG. 3 is a block diagram of an example of a photoacoustic imaging system that includes a photoacoustic device, such as one of the devices in FIG. 1 or 2.

### DETAILED DESCRIPTION OF THE INVENTION

The invention relates to photoacoustic (“PA”) imaging for intravascular applications, generally known as intravascular photoacoustics (“IVPA”). Disclosed here is an intravascular device capable of performing both frequency domain IVPA imaging and intravascular ultrasound (“IVUS”) imaging. Frequency domain IVPA is achieved using an intensity-modulated continuous wave (“CW”) laser to create PA images at the distal tip of the intravascular device.

The intravascular device includes an ultrasound transducer that is used to detect the photoacoustic energy generated by the intensity-modulated CW laser and thus create an image from within the lumen of a vessel or other organ. The ultrasound transducer can also be used to generate ultrasound images of the vessel or organ lumen. In general, the IVPA imaging device of the present invention can be used for imaging the lumen of a vessel or other organ; imaging atherosclerosis and plaque formations; characterizing atherosclerosis; and guiding interventional procedures, such as stent placements. It will be appreciated, however, that the IVPA imaging device can be employed for numerous other imaging and interventional applications. For instance, the IVPA imaging device can be used to image the interior or exterior surfaces of other bodily lumens and cavities of the body. Some examples of these other applications include imaging the prostate via the urethra and imaging the esophagus via the oral tract.

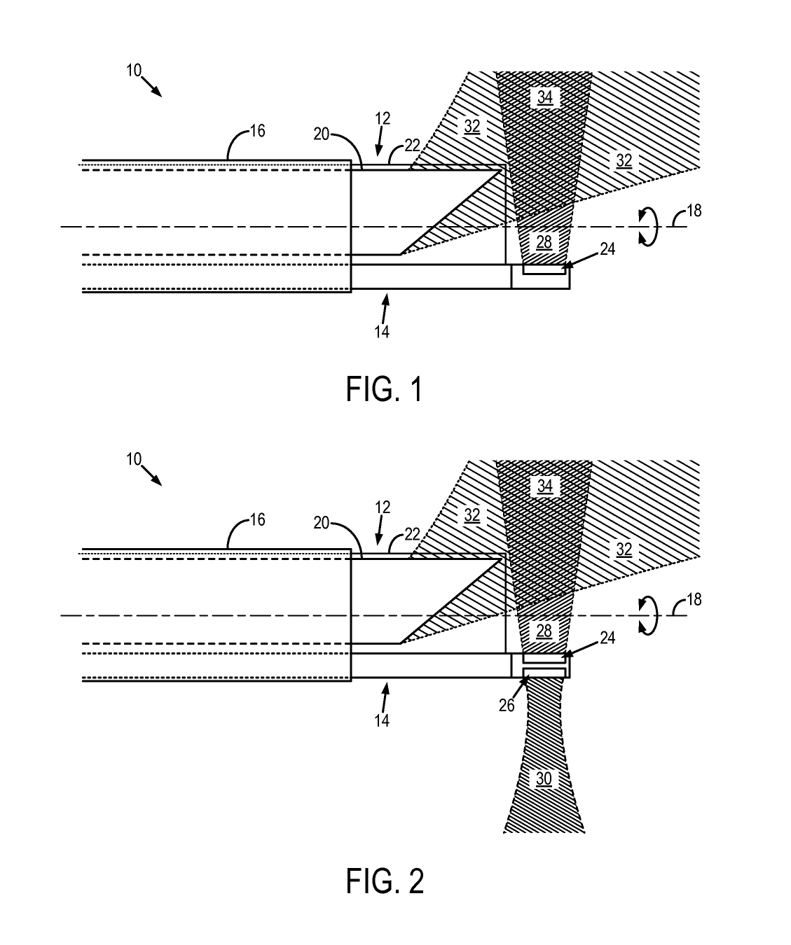

An example of an intravascular photoacoustic (“IVPA”) imaging device 10 of the present invention is illustrated in FIG. 1. The IVPA imaging device 10 is generally constructed as a catheter device that includes a fiber assembly 12 and a transducer assembly 14 that are coupled together. For instance, the fiber assembly 12 and transducer assembly 14 may be coupled via a common outer sheath 16 that holds the fiber assembly 12 and transducer assembly 14 in spaced arrangement. The fiber assembly 12 and transducer assembly 14 extend from the proximal end of the IVPA imaging device 10 towards the distal end of the IVPA imaging device 10 along a longitudinal axis 18 of the IVPA imaging device 10.

The fiber assembly 12 includes at least one optical fiber 20. At the distal end of the fiber assembly 12, the optical fiber 20 is preferably angled so as to transmit light outward at an angle from the longitudinal axis 18 of the IVPA imaging device 10. Optionally, an optically transparent protective cap 22 may be placed at the distal end of the fiber assembly 12 such that the optical fiber 18 is not in direct contact with the environment surrounding the IVPA imaging device 10, which may be an intravascular environment or other intraluminal environment with a bodily lumen or cavity. An illumination delivery mechanism delivers light to the distal end of the fiber assembly 12. In some embodiments, a source laser may be used to deliver light to the distal end of the fiber assembly 12. As an example, the source laser may be a CW laser or a pulsed laser that delivers light via the optical fiber 18. The optical fiber 18 and illumination delivery system are coupled at the proximal end of the fiber assembly 12.

The transducer assembly 14 generally includes an photoacoustic transducer 24 for receiving photoacoustic signals generated by an illumination field, such as an illumination field generated by pulsed or continuous wave laser light. In some configurations, the photoacoustic transducer 24 can also be operated to generate ultrasound energy and to receive pulse-echo ultrasound emissions. In this configuration, the photoacoustic transducer 24 would be operated in a receive-only mode for photoacoustic imaging and then, when the illumination field is not being generated, the photoacoustic transducer 24 could also be operated in an ultrasound imaging mode to obtain ultrasound images. In some configurations, the photoacoustic transducer 24 may include multiple transducer elements, some of which may be dedicated solely for receiving photoacoustic signals while others may be dedicated solely to generating and receiving pulse-echo ultrasound signals.

In some configurations, such as the one illustrated in FIG. 2, the transducer assembly 14 may include at least two transducers: a dedicated photoacoustic transducer 24 and a dedicated ultrasound transducer 26 for generating and receiving pulse-echo ultrasound signals. In this dual-transducer configuration, both photoacoustic and ultrasound images can be obtained. With the dual-transducer configuration, photoacoustic and ultrasound images can be obtained simultaneously and, even when not obtained simultaneously, are innately co-registered given the spatial relationship between the photoacoustic transducer 24 and the ultrasound transducer 26. This dual-transducer configuration thus provides a reduction in overall scan time.

As an example, the photoacoustic transducer 24 and ultrasound transducer 26 can be arranged to be oppositely facing, such as being rotated 180 degrees about a common longitudinal axis. In other examples, the photoacoustic transducer 24 and ultrasound transducer 26 can be arranged such that they are distributed about a common longitudinal axis by an angle greater than, or less than, 180 degrees. It is noted that the photoacoustic transducer 24 and ultrasound transducer 26 may include one or more transducer elements and may include arrays of transducers. In some embodiments, the center frequency of the ultrasound transducer 26 may be in the range of 20-40 MHz.

In the dual-transducer configuration, the photoacoustic transducer 24 and ultrasound transducer 26 are arranged at the distal end of the transducer assembly 14 such that the sensitive region 28 of the photoacoustic transducer 24 and ultrasound emission field 30 generated by the ultrasound transducer 26 extend in different directions so as to avoid interfering with each other. For instance, the photoacoustic transducer 24 and ultrasound transducer 26 may be arranged so as to be oppositely facing.

The IVPA imaging device 10 may be rotated about its longitudinal axis 18 through a plurality of different orientations such that cross-sectional imaging of the interior or exterior surface of a bodily lumen or cavity, which may include a vessel lumen, can be achieved. In other configurations, however, the fiber assembly 12 and photoacoustic transducer 24 (or both photoacoustic transducer 24 and ultrasound transducer 26) can be configured to provide 360 degrees of coverage. In these configurations, the illumination field 32 and sensitive region 34 would both span 360 degrees of coverage, thereby eliminating the need to rotate the IVPA imaging device 10 to obtain a full cross-section image.

In general, the distal end of the transducer assembly 14 may extend beyond the distal end of the fiber assembly 12 such that the sensitive region 28 of the photoacoustic transducer 24 is aligned with the illumination field 32 generated by the fiber assembly 12. This arrangement produces a field-of-view 34 in which photoacoustic signals are detected.

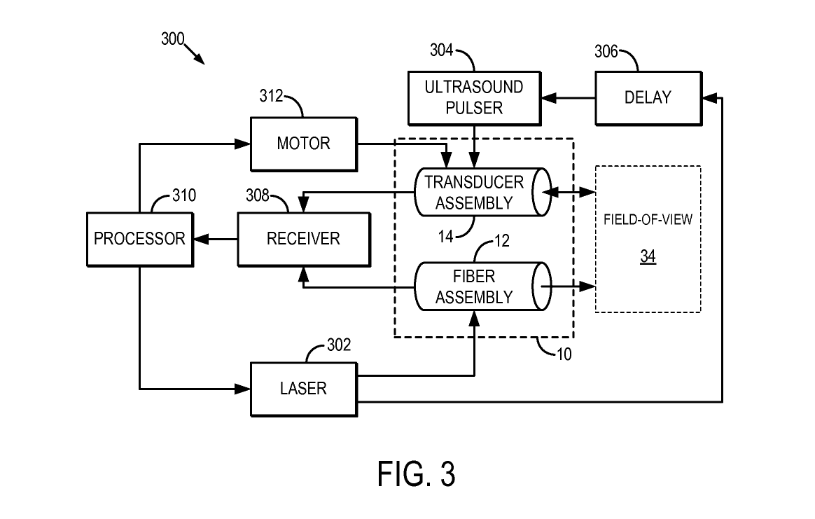

Referring now to FIG. 3, a block diagram of an example IVPA imaging system 300 that incorporates an IVPA imaging device 10 is illustrated. The IVPA imaging system 300 may include a laser source 302 that is a continuous wave laser, or in some configurations, that is a pulsed laser. The laser source 302 may include, for example, multiple laser systems or diodes, providing different optical wavelengths, that are fed into one or more laser fibers. The selection of an appropriate excitation wavelength for the laser source 302 is based on the absorption characteristics of the imaging target. Because the average optical penetration depth for intravascular tissue is on the order of several to tens of millimeters, the 400-2100 nm wavelength spectral range is suitable for IVPA applications. Thus, the laser source 302 may be, for example, an Nd:YAG (neodymium-doped yttrium aluminum garnet) laser that operates at 1064 nm wavelength in a continuous mode.

The laser source 302 is coupled to the fiber assembly 12 portion of the IVPA imaging device 10 at its proximal end, as described above. Irradiation with the laser source 302 is performed at a given point for a finite amount of time with an optical excitation waveform. In frequency-domain PA applications, in which a continuous wave laser is used, the optical excitation waveform is amplitude modulated with frequency sweeping, such as a chirp or pulse train. The irradiation produced by this type of optical excitation results in a frequency-domain photoacoustic modulated signal being produced in the region 32 illuminated by the IVPA imaging device 10. The chirp can include a multitude of different excitation waveforms including linear, non-linear, and Gaussian tampered frequency swept chirps.

Operation of the transducer assembly 14 may be controlled by an ultrasound pulser 304, which provides ultrasound excitation waveforms to the ultrasound transducer 26, and optionally the photoacoustic transducer 24 when configured to also transmit ultrasound. In single-transducer configurations in which the photoacoustic transducer 24 is used to both receive photoacoustic signals and to generate and receive pulse-echo ultrasound signals, a delay 306 between the laser source 302 and the ultrasound pulser 304 provides a trigger signal that directs the ultrasound pulser 304 to operate the photoacoustic transducer 24 at a delay with respect to the irradiation of the field-of-view 34. The timing provided by the delay 306 enables the detection of photoacoustic signals by the photoacoustic transducer 24 in the transducer assembly 14 when the field-of-view 34 is being illuminated, but also the generation and detection of pulse-echo ultrasound signals when the field-of-view 34 is not being illuminated.

Signals received by the transducer assembly 14 are communicated to a receiver 308, which generally includes a pre-amplifier, but may also include one or more filters, such as bandpass filters for signal conditioning. The received signals are then communicated to a processor 310 for analysis. The processor 310 can also control operation of a motor 312 used for rotating the IVPA imaging device 10. For instance, the motor may include a stepper motor that can be operated to incrementally rotate the IVPA imaging device 10 such that the transducer assembly 14 is able to generate photoacoustic and/or ultrasound pulse-echo signals from an entire cross-section of the imaging target.

Thus, the generated photoacoustic signals are detected by the photoacoustic transducer 24, communicated to the receiver 308, and then communicated to the processor 310 for processing and/or image generation. Similarly, pulse-echo ultrasound signals received by either the photoacoustic transducer 24 or a dedicated ultrasound transducer 26 can also be communicated to the receiver 308 and then communicated to the processor 310 for processing and/or image generation. This process is repeated in a radial format by rotating the IVPA imaging device 10 to image an cross-section, or radial view, of the vessel or organ lumen being imaged. Once a completed radial view is acquired with sufficient averaging, frequency-swept photoacoustic signals emitted due to the laser irradiation may be processed to perform depth profilometric imaging using digital signal processing techniques like matched filtering, or by means of a lock-in amplifier.

The present invention has been described in terms of one or more preferred embodiments, and it should be appreciated that many equivalents, alternatives, variations, and modifications, aside from those expressly stated, are possible and within the scope of the invention.

1. A photoacoustic imaging device, comprising:
a laser configured to generate laser light;
at least one optical fiber extending from a proximal end to a distal end along a longitudinal axis, the at least one optical fiber being optically coupled at its proximal end with the laser so as to generate an illumination field at its distal end;
a transducer assembly extending from a proximal end to a distal end along a longitudinal axis and being coupled, the transducer assembly comprising:
an photoacoustic transducer arranged at the distal end of the transducer assembly, the photoacoustic transducer being configured such that a field-of-view of the photoacoustic transducer is co-aligned with the illumination field and to receive photoacoustic emissions generated by the illumination field in the field-of-view.

2. The photoacoustic imaging device as recited in claim 1 in which the transducer assembly further comprises an ultrasound transducer configured to generate an ultrasound emission field that extends along a direction from the ultrasound transducer such that the ultrasound emission field avoids interfering with the sensitive region of the photoacoustic transducer.

3. The photoacoustic imaging device as recited in claim 2 in which the ultrasound transducer comprises more than one transducer element.

4. The photoacoustic imaging device as recited in claim 1 in which the photoacoustic transducer comprises more than one transducer element.

5. The photoacoustic imaging device as recited in claim 1 in which the distal end of the transducer assembly extends beyond the distal end of the at least one optical fiber.

6. The photoacoustic imaging device as recited in claim 1 in which the transducer assembly and at least one optical fiber are configured to rotate such that the field-of-view is rotated through a plurality of different angles.

7. The photoacoustic imaging device as recited in claim 6 in which the transducer assembly and at least one optical fiber are configured to rotate together such that illumination field and sensitive region of the photoacoustic transducer remain co-aligned while being rotated through the plurality of different angles.

8. The photoacoustic imaging device as recited in claim 1 in which the laser is a continuous wave laser that is configured to generate continuous wave laser light.

9. The photoacoustic imaging device as recited in claim 8 in which the continuous wave laser is configured to modulate the intensity of the continuous wave laser light.

10. The photoacoustic imaging device as recited in claim 1 in which the laser is a pulsed laser that is configured to generate pulsed laser light.

11. The photoacoustic imaging device as recited in claim 1 in which the transducer assembly and at least one optical fiber are sized to fit within a catheter that is configured to be introduced into a lumen of an organ.

12. The photoacoustic imaging device as recited in claim 11 in which the organ is at least one of a blood vessel, a urethra, and an esophagus.

13. The photoacoustic imaging device as recited in claim 1 further comprising a processor in communication with the transducer assembly and configured to receive photoacoustic signals from the photoacoustic transducer and to produce an image from the received photoacoustic signals.

14. The photoacoustic imaging device as recited in claim 13 in which:
the transducer assembly further comprises an ultrasound transducer configured to generate an ultrasound emission field that extends along a direction from the ultrasound transducer such that the ultrasound emission field avoids interfering with a sensitive region of the photoacoustic transducer; and
the processor is further configured to receive pulse-echo ultrasound signals from the ultrasound transducer and to produce an image from the received pulse-echo ultrasound signals.

15. The photoacoustic imaging device as recited in claim 14 in which the processor is configured to co-register the image produced from the photoacoustic signals with the image produced from the pulse-echo ultrasound signals.

16. The photoacoustic imaging device as recited in claim 1 further comprising a sheath disposed about the transducer assembly and the at least one optical fiber, the sheath coupling the transducer assembly and the at least one optical fiber.

17. A combined photoacoustic and ultrasound imaging device, comprising:
a fiber assembly comprising at least one optical fiber extending from a proximal end to a distal end along a longitudinal axis, the fiber assembly being configured to optically couple the proximal end of the at least one optical fiber to a light source so as to receive light and generate an illumination field at the distal end of the at least one optical fiber;
a transducer assembly coupled to the fiber assembly such that the transducer assembly and fiber assembly, the transducer assembly comprising:
an photoacoustic transducer configured to receive photoacoustic emissions generated in a field-of-view that is co-aligned with the illumination field; and
an ultrasound transducer configured to generate an ultrasound emission field that extends along a direction from the ultrasound transducer such that the ultrasound emission field avoids interfering with the field-of-view.

18. The combined photoacoustic and ultrasound imaging device as recited in claim 17 further comprising a light source that is optically coupled to the proximal end of the at least one optical fiber.

19. The combined photoacoustic and ultrasound imaging device as recited in claim 18 in which the light source is a continuous wave laser source.

20. The combined photoacoustic and ultrasound imaging device as recited in claim 18 in which the light source is a pulsed laser source.
